(19) United States
(12) Patent Application Publication
(10) Pub. No.: US 2010/0185299 A1
(43) Pub. Date: Jul. 22, 2010

(54) Bone Implant, and Set for the Production of Bone Implants

(76) Inventor: Berthold Nies, Frankisch-Crumbach (DE)

Correspondence Address: GUDRUN E. HUCKETT DRAUDT, SCHUBERTSTR. 15A, WUPPERTAL, 42289, DE

(21) Appl. No.: 12/516,354

(22) PCT Filed: Nov. 27, 2007
(86) PCT No.: PCT/DE07/02185

§ 371 Date: Feb. 7, 2010

(30) Foreign Application Priority Data
Nov. 27, 2006 (DE) 10 2006 055 892.8

(51) Int. Cl. A61F 2/28

(52) U.S. Cl. 623/23.53; 623/23.6

(57) ABSTRACT

The invention relates to bone implants and sets for producing bone implants. Said bone implants are made from pasty or cementitious preparations which are introduced into open-cell metal structures comprising an interconnected pore system as solid or porous materials and are optionally allowed to set, the metal structure per se being biocompatible in biological conditions and being stable or corrodible. The bone implant according to the invention contains at least one open-cell metal structure that has an interconnected pore system which is at least partially filled with a preparation made of at least one bone replacement material. The open-cell metal structure is significantly less rigid than the solid material made of the same metal.

The invention concerns bone implants and sets for producing bone implants. They are comprised of bone replacement materials on the basis of nano-crystalline calcium phosphates that, as solid or porous material, are introduced into open-cell metal structures with an interconnecting pore system and optionally cured, wherein the metal structure itself is biocompatible under biological conditions and may be stable or corrodible.

### BACKGROUND

As bone replacement materials for reconstruction of defective bone structures large quantities of biogenic bone materials are still used, i.e., body-own bone and donor bone, even though harvesting of such materials is connected with significant side effects and risks and they are in no way standardized Synthetic alternative products comply with the quality requirements of medicinal product laws or corresponding regulations outside of Europe but are often, rightly or wrongly, viewed as inadequate compared to biogenic materials. The quantitatively largest demand concerns a high-quality replacement material for donor bone that is in particular required for reconstruction of large and mechanically loaded bone defects as present in case of prosthesis exchanges. The result-determining properties of such a product are high mechanical stability (comparable to dense spongiosa up to corticalis), remodeling capability within the bone metabolism and integration into the bone structure, and stimulation of bone regeneration, and, last but not least, competitive manufacturing costs.

Products with a combination of the aforementioned properties are currently neither available on the market nor are such developments known in the literature. An expert however knows several bone replacement products that are partially or completely comprised of calcium phosphates but also may contain portions of other elements or their compounds, such as especially carbonate, silicate, fluoride, sulfate, magnesium, strontium, or alkali ions. Further components are in general contained in very small amounts and are to be viewed as contaminants. Such preparations are available as solid or porous shaped bodies or in granular form or, recently, also increasingly available as cement-like preparations that during surgery are mixed from powder and liquid to a paste and after introduction into the bone defect cure within a relatively short period of time.

Independent of the physical form, all known products of this kind (calcium phosphate cements, porous shaped bodies and granular material) are not satisfactorily and physiologically mechanically loadable and accordingly are not approved and/or suitable for load-bearing applications. Sintered solid calcium phosphate ceramics have a very high compression strength (approximately 200 MPa for HA, cortical bone has approximately 150 MPa compression and flexural strength) but are at the same time very hard and brittle and have thus a very high stiffness that surpasses that of bone (module of elasticity approximately 100 GPa for HA in contrast to maximum of 20 GPa for cortical bone and maximally approximately 1.5-3 GPa for spongiosa (upper value corresponds to healthy vertebra)), in particular that of the trabecular or spongiosal bone, and therefore deviates greatly from the biomechanics of the bone. Moreover, such materials have only minimal bioactivity and are usually not resorbable.

In contrast to this, some granular material, shaped bodies and cement-like preparations of calcium phosphate that are available on the market and usually have reduced strength are bioactive and resorbable, i.e., in the ideal situation these materials are integrated into the bone metabolism and in the end are replaced by new bone (remodeling).

Where in orthopedics, trauma surgery, vertebral surgery, oral and maxillofacial surgery, and neurosurgery mechanically highly loaded defects must be stabilized and filled, the user relies on metal implants (tensile strength usually >1,000 MPa and modulus of elasticity >100 GPa (TiAlV)) and uses often donor bone as an (inexpensive) mechanically loadable fill and repair material that as a solid material has the strength values of corticalis.

Recently, first for technical applications open-cell metal structures have been developed whose suitability has been examined also for bone implants in the meantime. Such product has been offered on the market by the orthopedic company Zimmer (formerly; Implex) under the name “trabecular metal” (formerly Hedrocel) as a prosthesis coating as well as exclusive implant material on the basis of the biocompatible metal tantalum. DE 31 06 917 also discloses “an implant as a replacement for spongiosal bone, characterized by a shape-stable open-pore or open-cell shaped body with spongy structure”. Corresponding open-pore implant structures are commercially offered by the company ESKA (Lübeck) as individual implants or as open-cell coatings on solid base materials. A combination of bone replacement materials or a utilization for reinforcement of bone replacement materials is however not described.

All those structures are considered open-cell metal structures in which metals form a scaffold structure and the remaining cavities (pores) are filled with another material, in general air or gas. The configuration of the pore system can vary greatly and may span a range from completely open-cell systems (as in the case of “trabecular metal”) with a structure that is reminiscent of spongiosal bone, hollow sphere structures with remaining interconnected pore system, to closed-cell structures (see CellMet-Conference, Dresden, 2005), as further literature regarding the prior art compare Adler et al., “Sintered Open-Celled Metal Foams Made by Replication Method—“Manufacturing and Properties on Example of 316L Stainless Steel Foams”, in Cellular Metals and Polymers 2004, edited by R. F. Singer, et al. and John Banhart, “Manufacture, characterisation and application of cellular metals and metal foams”; in Progress in Materials Science 46 (2001) 559-632.

For use as bone implants primarily the open-cell materials with spongiosal structure and the hollow sphere structures with remaining pore system are of interest. The great advantage of open-cell metal structures is their high level of adaptability of mechanical properties at relatively minimal density. By artful selection of cell structures (pore size, web thickness, thickness of the spherical shells, pore distribution, material selection), almost all relevant strength parameters can be adjusted to values that provide the bone to be repaired with optimal mechanical repair and regeneration conditions.

The disadvantage of the available metals is however that in the best case they are satisfactorily biocompatible and thus do not lead to unacceptable rejection reactions. A satisfactory bioactivity that stimulates the bone to integrate the open-cell metal implant in that it grows into the pore system does not exist in case of the metals.

In conventional metal implants that are provided for permanent attachment in the bone, for example, uncemented joint prostheses, an increased bioactivity can be achieved in that their surface is coated with calcium phosphates and/or bioactive molecules (for example, adhesion peptides, collagen, ECM components, morphogenic proteins etc.). But also such bioactive metal structures correspond only unsatisfactorily to the physiological requirements of bone regeneration or do not respect satisfactorily the biological mechanisms of bone remodeling and are not capable of completely utilizing the regeneration potential of the body for rebuilding larger bone defects. Also, the aforementioned coatings in principle cannot provide a contribution to the mechanical reinforcement of the metal structure. This reinforcement however upon further consideration is of special importance for many fields of application because, on the one hand, the mass of metal to be implanted should be kept as small as possible this holds true for metal alloys that have long-term stability as well as particularly for bio-corrodible metals) and, on the other hand, the initial stability of the composite implant with respect to an early full load capacity should be as high as possible.

### PRIOR ART

DE 198 58 579 A1 discloses a bone replacement implant or interstitial vertebral implant that (expressly) is comprised of rigid metal foam and whose pores are filled with a foreign substance. As foreign substances bone cement, spongiosal material and plastic material are mentioned. The claimed pore sizes of the metal foam comprise the range of 0.5 to 5 mm and as materials titanium and titanium alloys TiAl6V4, TiNb6Al7, TiAl5Fe2,5, cobalt alloys and steel alloys according to ISO 5832-9 are mentioned. The main aspect of this patent application is apparently the improvement of an intervertebral implant with respect to radiological diagnostics. In this context, expressly an implant of a rigid metal foam is described and claimed that, despite the reduced amount of required material, has a very high stiffness. An adaptation of the mechanical properties of the metal foam to biomechanical requirements of the bone and in particular a reduction of the stiffness to values that are significantly below those of corresponding solid metal (metal alloy) is not mentioned. According to DE 198 58 579 A1 in principle the stiffness (or rigidity) of the solid metal is to be maintained and the porosity of the metal foam is to be utilized only for reduction of material in order to reduce artefact formations and visibility impairment in MRI and X-ray methods. Accordingly, it is expressly noted that the porous implants provide the same support function as solid implants.

DE4101526 A1 discloses a bone replacement that is “characterized in that the bone replacement is present in form of shaped bodies that are composed of two material components of which the first material component “strength” ensures a mechanical load capacity of greater than 1,000 N/cm² while the second material component “integration” has for stimulating the osteo-conductive effect a specific surface area of greater than 1.5 m²/g. Even though neither in the claims nor in the specification a more detailed material composition of the bone replacement is disclosed, it can be taken from the description that the inventor exclusively takes into consideration bioceramic components for the material component “strength”. Metal sponges or metal foams are not mentioned and particular none that, with regard to stiffness, are matched to the stiffness of bone.

DE 10 2005 018 644 A1 discloses an implant, webs therefor for treatment of defects of long bones, as well as a method for producing the implant. In this patent application, porous structures are disclosed that can be processed by joining at least two layers of web material to a porous bone implant for long bone defects. The implant with respect to form and function is primarily designed to serve as an areal support for culturing bone cells in order to be matched, after completion of cellular colonization, by multi-layer arrangement in long bone defects. Load-bearing applications as with the inventive implant material cannot be realized with the implant materials disclosed in DE 10 2005 018 644 A1.

DE 10 2004 016 874 B4 discloses a composite material for technical applications that is comprised of a nonmetallic inorganic matrix and a three-dimensional metallic network that is integrally connected therewith. For this purpose, the network is filled with the matrix materials or their precursors and matrix material and network are exposed to a temperature increase >600° C. Even though as a possible application the use as bone implant is mentioned, the described material and in particular the aforementioned manufacturing process is entirely unsuitable for development and production of bone implants with the aforementioned properties. The high temperatures required in this case for manufacture lead inevitably to a strong sintering effect of the aforementioned matrix materials (hydroxyl apatite, TCP) so that the bioactivity (that goes hand in hand with the nano structure) is massively reduced. Further disadvantages in case of the sintered hydroxyl apatite as a matrix is the lack of resorption capability, the undefinable composition of the integral connection of the metal and matrix (that in case of medicinal application is to be viewed as very critical), and the lack of a possibility for biomechanical adaptation (the sintered product according to the claimed manufacturing process always results in a material with extremely high stiffness that, as in the case of sintered hydroxyl apatite is a multiple of that of bone). The aforementioned material has therefore in relation to bone implants no advantages but instead rather disadvantages in regard to the older prior art (solid metal implants).

### OBJECT

The goal of the present invention is therefore to provide a bone replacement material that, on the one hand, has the mechanical performance and in particular the great mechanical adaptability of cellular metal structures to biomechanical requirements of the bone regeneration and, on the other hand, utilizes the regeneration-stimulating potential of nano-structured bone minerals.

The present invention has the object to provide a bone implant as well as a set for producing a bone implant that, on the one hand, has biomechanics matched to that of bone and immediately after implantation into the bone can be mechanically fully loaded and, on the other hand, fully utilizes the bone-stimulating potential of nano-crystalline bone minerals or their synthetic analogues.

### SUMMARY OF THE INVENTION

According to the invention, the object of the invention is solved by a bone implant according to the features of claim 1 as well as a set in accordance with claim 19. Further embodiments are contained in claims 2 to 18 as well as 19 to 23.

The bone implant according to the present invention contains a combination of an open-cell metal structure with a preparation of at least one bone replacement material (of nano-crystalline (analogues of) bone minerals). With this combination the possibility is provided for the first time of combining a bane replacement structure with high structural biocompatibility (i.e., biomechanical compatibility with the bone structure at the site of implantation) and simultaneously a high bioactivity, as is characteristic of nano-crystalline bone material. For this purpose, the bone implant according to the invention contains at least one open-cell metal structure with an interconnected pore system that is at least partially positive-lockingly filled with a preparation of at least one bone replacement material, wherein the stiffness of the open-cell metal structure is significantly lower than that of the solid material comprised of the same metal. Preferably, the stiffness of the open-cell metal structure surpasses the stiffness of healthy cortical human bone by not more than a factor of 2.

Examples of open-cell metal structures are the products “trabecular metal” of the orthopedic company Zimmer, the porous bone implants of the orthopedic company ESKA, open-cell metal structures of the firm m-pore, different experimental powder-metallurgically produced open-cell metal structures of the Fraunhofer Institut IFAM, and metal structures of the Fraunhofer Institut ILT that are produced by laser sintering.

According to the invention, open-cell metal foams are used that are adjusted by appropriate material selection, adjustment of porosity, pore size, web thickness etc. to stiffness values that are significantly below (>factor 2, preferred >factor 5, especially preferred >factor 10) that of solid metals and in which the upper limit of stiffness is limited to values that surpass by not more than the factor 2 the stiffness of healthy cortical bone in the human body. In general, the adjusted stiffness values will be significantly lower because the bone implants according to the invention are used primarily in spongiosal bone and accordingly are adjusted to the lower stiffness values of spongiosal bone.

A main function according to the invention of the employed metal foams is according to the present invention not only the support function for the bone itself but also the reinforcement (augmentation) and optionally shaping of bone replacement materials, whose own biomechanical properties are not satisfactory for the envisioned application. The limitation of the stiffness of the metal foam to values that are within the range of healthy bone (with a sufficient buffer for possible application errors during implantation or product selection) ensures that, on the one hand, the bone implant (of metal foam and nano-structured bone replacement material) during the implantation and during healing can be loaded in accordance with the medical indication and, on the other hand, during the further course of bone remodeling an extensive physiological mechanical stimulation of the treated bone defect is enabled while in this time period the nano-structured filler material is resorbed and replaced with new bone. A rigid metal foam would in this case lead to a distinct “stress shielding”, i.e. impair the desired bone formation within the pore system and would be counterproductive in this context of the present invention.

The introduced bone replacement material can significantly contribute to the initial stiffness of the composite bone implant and can determine the initial stiffness particularly in implants for high mechanical loads in a decisive way. This initial stiffness is reduced during the course of resorption of the bone replacement material down to the value of the metal foam (in bio-corrodible metal foams in the end down to zero) wherein then the ingrown bone contributes its part to the strength and structural stiffness.

The detailed shape and composition of the open-cell metal structures and of the introduced bone replacement materials can be selected in various ways in order to tailor them in a targeted fashion to the clinical application purpose. Important for the open-cell metal structures is however in any case an open interconnected pore system which in case of individual implants penetrates the entire implant and in case of assembled implants penetrates the cellular component. The latter applies for example to prostheses that comprise surface layers of cellular metal structures or to implants of a modular construction that contain components of cellular metal structures. Additional criteria for the cellular metal structure is a stiffness that can be adjusted to the values of the target bone and, with respect to its maximum value, surpasses by not more than a factor of 2 the stiffness of healthy cortical bone.

The open-cell metal structures are filled with preparations of primarily mineral bone replacement materials so that the bone replacement materials during storage, transport, and implantation are safely fixed in the open-cell metal structure and can be used easily.

The present invention fulfills two aspects.

On the one hand, the mechanical stabilization of a bone defect is ensured by the open-cell metal structure insofar as the pore system is loaded with a filler material that is optimized with respect to a bone-stimulating effect, wherein the bone replacement material is loadable only minimally, or particularly, with regard to compression while the open-cell metal structure may also absorb and primarily absorb tensile loads. The composite material can thus assume properties comparable to those of steel-reinforced concrete where the concrete matrix also determines the compression strength and stiffness and the steel reinforcement improves the tensile strength.

On the other hand, by means of the composite of open-cell metal structure and bioactive cement-like filler, mechanical properties are achieved that cannot be obtained with any of the individual components alone. In particular, by a targeted selection of metal structure and bone replacement material a strength course can be adjusted over the temporal course after implantation that adapts in a medically and biologically desired way to the changing requirements of the dynamic bone structure. For this purpose it is required that the bone replacement material is continuously replaced with bone in that it is integrated into the bone metabolism and is subjected to the cellular regulation mechanisms of bone restructuring (remodeling). In case of a permanently stable metal, the latter will support even after resorption of the bioactive resorbable and mechanically loadable (with regard to compression) filler a portion of the biomechanical load while the grown-in bone, according to Wolff's law, is excited by means of the mechanical stimulation to build itself up until is can support the remaining load portion. Also in this respect, a minimization of the metal proportion of the composite implant is desirable in order to minimize the so-called “stress shielding” in the interior of the implant.

According to the invention the stiffness of the open-cell metal structure is to be adjusted to values with respect to medical indication that are particularly beneficial for the mechanical stimulation of the regeneration of the bone at the implantation site. As an upper limit of the stiffness for the cellular metal structure a value at the level of healthy cortical bone is therefore viewed as satisfactory but with respect to safety in regard to faulty applications and with respect to the changing stiffness of bio-corrodible metals an upper limit is assumed that surpasses the value of healthy cortical bone by not more than the factor 2. This value is still significantly below the value for the most important relevant implant metals in dense form. In practice, the stiffness values of the cellular metal structures are adjusted to significantly lower values because the implants according to the invention are used primarily as replacement for spongiosal bone and, as bone replacement materials, nano structured calcium phosphate preparations are used that, in turn, contribute significantly to the initial structural stiffness of the implant. After completed resorption of the bone replacement material a drop in stiffness to values below the surrounding bone is indeed desirable. For bio-corrodible metals a further aspect resides in that even for biocompatible metals the quantity of degradation products should be kept as small as possible in order to not be subject to narrow limitations especially with respect to the required volume of the implant (for those products that are conceptually provided primarily for large volume bone replacement).

According to an advantageous embodiment of the invention, the open-cell metal structure is comprised of a biocompatible metal. For this purpose, the open-cell metal structure may be comprised of Nitinol or titanium, tantalum, magnesium, iron, cobalt, niobium, rhenium, hafnium, gold, silver or their alloys with one another or with other elements, wherein these alloys contain the aforementioned elements to at least 60% by weight respectively.

The open-cell metal structure can be comprised principally of a metal or its alloys that under biological conditions is permanently stable or bio-corrodible. In the case of permanently stable metals they are comprised preferably of stainless steel, alloys based on cobalt, pure titanium, titanium alloys, Nitinol, tantalum, tantalum alloys, niobium, gold, silver. In case of bio-corrodible metals or their alloys those metals are preferred whose corrosion products produced under biological conditions are comprised of compounds whose components occur naturally within the body of vertebrates, in particular iron or alloys of iron or magnesium as determinative alloying elements.

According to a further advantageous embodiment of the invention the open-cell metal structure is coated with a further metal that is not a component of the alloy, or an inorganic nonmetallic or organo-mineral material.

A prerequisite for the rejection-free healing of bone implants is the biocompatibility of the employed materials. As especially compatible metallic materials for the implantation in direct bone contact (for permanent implantation) over the last few years titanium and its various alloys (for example, Ti6Al4V, Ti5Al4Nb Ti5Al2,5Fe, Nitinol) have been found to be suitable. Also very well compatible are a few further metals such as tantalum, niobium, molybdenum, rhenium, hafnium, and their alloys. However, stainless steel alloys and cobalt-based alloys are still used in large quantities that are provided partially with coatings for improvement of biocompatibility that are to prevent diffusion of (toxic) metal ions (for example, silicon nitride). The currently employed metals and alloys for bone implants as well as those that are taken into consideration especially for manufacture and use as open-cell metal implants for use in bone as well as their coated and uncoated variants are considered for the inventive combination with resorbable bone replacement materials in as much as they fulfill the mechanical prerequisites which is the case in particular for (stainless) steels, titanium alloys and cobalt alloys. The other aforementioned metals are considered in particular as alloying partners or coatings but in case of tantalum can also be the main component of an alloy. In addition, also further materials and modifications are considered that are currently less common but have already been tested in research for use as bone implants. Special mention should be made of bio-corrodible metals such as in particular iron-based and magnesium-based alloys as well as metals that are coated with silver or other anti-infective substances for preventing foreign body-associated infections.

The open-cell metal structure is thus comprised of a biocompatible metal or a metal alloy that is either permanently stable (for example, implant steel, cobalt-based alloys, titanium/titanium alloys, tantalum, niobium, Nitinol, rhenium, hafnium, gold, silver etc.) or is corrodible in a physiologically acceptable way after implantation in the body (with release of body-compatible degradation products) (for example, magnesium/magnesium alloys, iron/iron alloys, zinc/zinc alloys etc). It is characterized by a porous structure in which substantially all pores (>90% of the pores) form an interconnected porous system (in the case of metallic hollow sphere structures this concerns only the pores between the spheres and not the cavities in the spheres) and by a stiffness that is significantly below that of solid metals and with respect to its maximum value surpasses by not more than the factor 2 the stiffness of healthy cortical bone. In this connection, the shape and size of the pores or cells as well as the thickness of the webs or sphere shells can be varied in a targeted fashion within the implant in order to obtain in this way a graduated structure.

In a special embodiment of the bone implant according to the invention the optimal mechanical adaptation of the implant to the conditions at the implantation site is achieved by a targeted control of the porosity in that pore diameter, pore shape and/or pore volume is changed in at least one section plane of the implant or the open cell component of an implant.

A graduated structuring mimics the biological construction of the bone and enables in combination with the bone replacement material and its reinforcement in a unique way (different from solid implant materials and those with purely random pore arrangement and pore structure) new degrees of freedom with respect to design of targeted biologically and biomechanically adapted implants for the repair and the regeneration of large bone defects.

A further aspect of the bone implant according to the invention is that the preparation contained in the bone implant is comprised of at least one bone replacement material comprising an osteo-conductive or osteo-inductive or osteo-genetic property or a combination of these properties and having a bone growth stimulating effect under implantation conditions. The preparation of at least one bone replacement material can be a mineral filler or organo-mineral filler that fills the interconnected porous system of the open-cell metal structure macroscopically homogeneously wherein the bone replacement material itself preferably may contain pores (micro pores and nano pores) that are substantially uniformly distributed about the entire implant or, as in the case of the pore system of the metal structure, are also embodied in a graduated fashion. The preparation of at least one bone replacement material is comprised preferably, relative to the dry matter, to at least 30% by weight of calcium phosphates. The bone replacement material contains nano-crystalline calcium phosphate or at least one bone replacement material that forms after introduction into the body nano-crystalline calcium phosphate.

The chemical composition of the preparation of at least one bone replacement material mimics the mineral phase of natural bone and is comprised primarily of calcium ions and phosphate ions that as further components may contain in particular carbonate ions, silicate ions, fluoride ions, sulfate ions, magnesium ions, strontium ions, zinc ions, iron ions and alkali ions as well as oxides and in which traces of further inorganic compounds may be present. A component of the preparation of at least one bone replacement material may be quantitatively minimal proportions (<50% by weight) of organic compounds, such as in particular collagen, gelatin, other proteins, glycoproteins, peptides, amino acids and their derivatives, monosaccharides, oligosaccharides, and polysaccharides, vitamins, citrates, surface-active agents, buffering substances, biocompatible synthetic polymers and generally body-compatible organic compounds that may have an effect on strength, cohesion, and the micro structure and nano structure of the mineral phase.

The preparation of at least one bone replacement material is present in the pore system of the open-cell metal structure as (compacted or bonded) powder, pasty suspension (for example, as a nano-crystalline hydroxyl apatite suspension in aqueous solution or non-reactive pasty suspension, as realized, for example, in the product Ostim), lyophilisate or cement-like cured material inasmuch as it fulfills the requirement that during storage, transport, and implantation its composition and structure will not change in a disadvantageous way (for example, falling out of the pore system). An important aspect of the filler is its resorption capability under physiological conditions that is a function of its composition and crystal structure.

Characteristic for the introduced, primarily mineral, bone replacement materials is that they fill the pore system of the open-cell metal structure in such a way that a high bonding capacity for bone-active biomolecules as well as also an intensive material exchange with the surrounding medium and excellent accessibility for the bone cells are ensured. The limitation (primarily mineral) concerns the fact that nano-crystalline bone replacement materials of a purely mineral composition and homogenous macrostructure in principle are already capable of, in the context of the invention, stimulating bone growth in that they bind and accumulate biomolecules contained in the serum that are important for the bone metabolism and make them available for the bone metabolism and, at the same time, function as a degradable substrate for the osteoclasts. During this degradation, the biomolecules that are bonded to the bone minerals or their bioactive fractions as well as signal compounds synthesized by the osteoclasts for stimulating differentiation and activity of the osteoblasts are released.

The calcium phosphate cements of components of the aforementioned compositions have partially very different mechanical properties. Their compression strengths are usually in the range between approximately 5 and 100 MPa. The flexural strength of the materials that are not augmented is very minimal. For generating a compositional strength between open-cell metal structure and the cement-like resorbable bone replacement materials, it is required that both components are connected to one another across extended areas in a positive-locking way in order to ensure that the introduced forces can be transmitted onto the other material, respectively.

For obtaining a high bioactivity, it is necessary that the nano-structured bone replacement materials as a bioactive filler have a high specific surface area. Specific surface area and nano structure are correlated directly with one another, i.e., the finer the nano structure, the greater the specific surface area. Desirable is a specific surface area for the bioactive filler of >1 m²/g, preferably >5 m²/g, especially preferred >25 m²/g and most preferred >50 m²/g. This high specific surface area is preferably achieved by precipitation reactions under biomimetic conditions because high temperatures lead to a strong reduction of the specific surface area. Preferred synthetic conditions for the bone replacement materials are therefore in the range of natural conditions of the bone, in particular near body temperature. The bone replacement material are therefore also defined in that the structure-determining manufacturing steps for the nano-structured calcium phosphates as components of the bone replacement materials are produced at temperatures of <250° C., preferably <150° C., especially preferred <100° C. and most preferred <80° C.

As bone replacement materials, mineral bone cements on the basis of calcium phosphates and/or magnesium phosphates are preferred. On the one hand, they form typically upon curing nano-structured calcium phosphate phases as they are required for a high bioactivity and, on the other hand, they can be influenced in wide ranges with regard to their compression strength and porosity and therefore can contribute significantly to the mechanical strength of the implant materials. In the literature, numerous compositions of calcium phosphate cements (CPC) without and with various additives are described that are suitable for the inventive combination with the open-cell metal foams. Especially preferred are CPCs that after the curing reaction are comprised of hydroxyl apatite or calcium-deficient hydroxyl apatite. These CPCs can also be produced from various starting materials. As especially preferred versions compositions are considered that are comprised of α-TCP or β-TCP, CaHPO₄, CaCO₃, and precipitated hydroxyl apatite and that are mixed with water or aqueous buffer solutions to cements. Especially preferred are cements whose powder component is comprised to >50% of α-TCP or β-TCP. Even more preferred among this group are cements that contain α-TCP and/or β-TCP in quantities of more than 50% in the powder mixture and contain further calcium salts in a mixing ratio that produces in the powder mixture a calcium/phosphate ratio between 1.3 and 1.5. Also preferred are cements that, as a powder component, contain tetra calcium phosphate (TTCP) and/or dicalcium phosphate (CaHPO₄, DCPD or DCPA). Especially preferred among this group are cements that contain TTCP and DCPD or DCPA in a mixing ratio that provides in the powder mixture a calcium/phosphate ratio between 1.5 to 1.8. Preferred fillers are also CPCs that produce upon curing as a cured product DCPD (brushite) and have a calcium/phosphate ratio of approximately 1.0.

In addition to the CPCs there are also further nano-crystalline calcium phosphate preparations (nano HA) known in the literature, and methods are described that lead to nano-crystalline calcium phosphates. As bone replacement materials according to the invention all precipitated calcium phosphates are suitable that have a specific surface area of >1 m²/g, preferably >5 m²/g, in particular >25 m²/g and especially preferred >50 m²/g and whose calcium/phosphate ratio is in the range of 1.35 to 1.8 (preferred 1.4-1.7). These calcium phosphate preparations are present as suspensions in water or aqueous solutions and can be used in this form (see examples 1, 3, 5, 7). Preferred preparations have a solids proportion of >10%, preferred >20% and especially preferred >30%. Also particularly preferred are nano-HA preparations with comparable solids contents that, in addition to the nano-crystalline calcium phosphates, also contain further components such as proteins (for example, collagen or gelatine). Also especially preferred are preparations that are obtained from such preparations by drying, freeze-drying or exchange of the suspension medium.

For increasing the biological activity of purely mineral bone replacement materials successful experiments have been performed with compositions that contained in addition to the mineral components one or several organic substances. As organic components in particular collagen and its derivatives (for example gelatine, P15), other proteins of the extra-cellular matrix (ECM proteins, for example, fibronectin), synthetic adhesion peptides (RGD peptides), polysaccharides (hyaluronic acid, chondroitinic sulfate, chitosan, starch, cellulose, and their derivatives, respectively), morphogenic proteins (BMPs, especially BMP2 and BMP7, TGF-β), angiogenic growth factors (bfGF, VEGF), vitamins (C, B, E, D), and small organic molecules (citrate, surface-active agents, salts of the glycerophosphoric acid, amino adds and their derivatives) are used. These organic components have partially on the one hand the effect that they beneficially affect the nano-crystallinity of the mineral component (in the sense of a finer structure), and, in this way, increase the adsorption capability for bone active biomolecules that, in turn, indirectly increase the bioactivity of the mineral phase. On the other hand, they can affect directly the differentiation and activity of the bone cells as is the case in particular for the growth factors, morphogenic proteins, adhesion peptides and ECM proteins. The above list may be further expanded; however, with respect to the present invention this is not important. It is instead important here that the bioactivity of bone replacement materials according to the invention can be affected in numerous ways and that such combinations are included in the invention inasmuch as they are combined with open-cell metal structures in the way described.

The biological activity of bone implants or bone replacement material can be divided into three aspects: osteoconduction, osteoinduction, and osteogenesis that each have a specific biological basis. For the bone implants according to the invention the osteoconduction is of great importance because it is already a characteristic property of the primarily employed nano-crystalline calcium phosphates. Osteoconductive properties are however not limited to calcium phosphates but can also be detected for other material classes, such as glasses, polymers or other ceramics (than those based on calcium phosphate).

Osteoinduction, induction of cell growth and osteodifferentiation to bone tissue (also outside of the bone), is a typical property of morphogenic proteins (such as in particular BMP 2 and BMP7 but also of other representatives of the TGF-β superfamily). Recently, however osteoinductive effects have been detected also (again) in purely mineral materials and in particular in case of bi-phase calcium phosphates that are comprised of hydroxyl apatite and β-tricalcium phosphate. It is to be expected that further materials with osteoinductive properties will be identified.

Osteogenesis, formation of bone tissue by differentiated bone cells or precursor cells or stem cells that have been caused to differentiate (also outside of the bone), is viewed in addition to osteoinduction as a key for treatment of large or complex bone defects. Osteogenisis is the basis for bone-tissue engineering.

All three aspects can be realized or combined in an advantageous way with the hone implants according to the invention. Osteoconductivity is an integral component or property of the (organo) mineral filler, while the osteoinductivity may be accomplished by use of hi-phase calcium phosphates or the addition of inductive active agents (see above) to the filler. In regard to combining calcium phosphate cements with morphogenic proteins, research results are already available (Transforming growth factor-1 incorporated during setting in calcium phosphate cement stimulates bone cell differentiation in vitro, E. J. Blom, J. Klein-Nulend, C. P. A. T. Klein, K. Kurashina, M. A. J. van Waas, E. H. Burger. Journal of Biomedical Materials Research, Volume 50, Issue 1, Pages 67-74 Published Online: 24 Jan. 2000). The combination of osteogenesis with the inventive bone implants is an especially attractive aspect for the treatment of very large bone defects with the methods of tissue engineering. The bone implants according to the invention serve in this connection as mechanically loadable support materials for the tissue culture that at the same time provide an osteoconductive matrix for culturing the bone cells and, as needed, can also be mechanically stimulated. Primary, directly after implantation they can be subjected to full loads and in this way long immobilization times, typical for all prior approaches of bone tissue engineering, can be avoided.

The preparation of at least one bone replacement material is comprised advantageously of a composite of bone-analog minerals and (structure) proteins or other (structure) polymers in which thus the organic component of the bone replacement material also contributes to the mechanical properties of the bone replacement material. Examples are in particular collagen, gelatin, chitin/chitosan (and derivatives), cellulose (and derivatives), starch (and derivatives), hyaluronic acid, chondroitic sulfate, and synthetic polymers, that alone or in various combinations have already been disclosed as implant materials for bone. In the context of the invention and also in accordance with the natural example of bone, the organo mineral fillers to a large extent are comprised of bone-analog minerals or mineral components from which bone-analog minerals after implantation in the body can form spontaneously. They include calcium phosphates (in particular α- and β-tricalcium phosphates and dicalcium phosphates), as well as magnesium phosphates, magnesium carbonates, silicates and/or sulfates of alkali or earth alkali elements or ammonium compounds, alone or in combination. All aforementioned substances are known as components of bioactive bone replacement materials and bone fillers. The presented list is not meant to be limiting in any way in view of the numerous combination possibilities but is to include all mineral components which in connection with the present invention, directly or indirectly, may be used for forming or precipitation of bone-analog minerals.

The quantity ratios of protein or polymer to mineral components, as a function of the combination partners, can be selected in wide ranges. Preferably, combinations are used that, relative to the dry matter, are comprised to at least 30 weight percent of calcium phosphate and/or collagen and/or other proteins of the extracellular matrix. The remaining proportion is comprised of further organic or inorganic substances (inclusive of possible active ingredients) that enhance the mechanical and/or biological activity of the organo-mineral bone replacement materials.

According to an advantageous embodiment of the invention the preparation comprised of at least one bone replacement material contains biologically and/or pharmacologically active ingredients. The release of pharmacologically and/or biologically active ingredients from the bioactive bone replacement material can be controlled in a targeted fashion by the composition and structure of the bone replacement material. In this respect, the product according to the invention can also be matched much better to clinical requirements than is possible with the individual products.

Examples of expedient pharmacologically active ingredients are antibiotics and other active ingredients with antimicrobial effect (antiseptic substances, antimicrobial peptides etc.) that are capable of healing already existing infections or that enhance the measures with respect to their treatment or are capable of counteracting prophylactically the generation of bone infections. This is in particular of great clinical importance for the predominant use of the product according to the invention in large bone defects because in these cases a comparatively high infection risk exits.

Other expedient, pharmacological active ingredients are substances that are capable of temporarily suppressing inflammation reactions in the environment of the implant so that an unimpaired bone healing may occur. Included are here combinations of the bone implants according to the invention with all active ingredients that are capable of suppressing in a specific or unspecific way inflammation reactions and in particular those that impair directly the acid secretion of inflammation cells.

Basically, as suitable active ingredients all substances are possible that are capable of assisting the primary goal of the implant, the stabilization and regeneration of the treated bone defect, and of minimizing the occurrence of undesirable events and processes that may be correlated with the specific clinical situation.

An optional addition of pharmacologically active ingredients, in particular those with bone-stimulating, antimicrobial, and inflammation-inhibiting function, in a concentration that is suitable to release the active ingredient over a clinically relevant period of time in an effective concentration therefore has in particular the function to enhance the effect of the implant in such cases where the conditions for a normal bone healing are impaired.

An advantageous embodiment of the invention in accordance with claim 14 resides in that the bone replacement material is porous. It is also advantageous, according to claim 15, that the preparation comprised of at least one bone replacement material fills the accessible pore volume of the pore system of the open-cell metal structure, relative to the theoretical/calculated possible degree of filling and calculated based on the dry matter, to 5 to 80 weight percent.

Instead of homogeneously constructed bone replacement materials in particular for filling larger bone defects, the use of porous materials has been found to be advantageous for the faster integration or penetration and resorption. Porosity increases the available surface area and facilitates thus the adsorption of serum components as well as the resorption capability by means of the larger accessible surface area for the osteoclasts. A disadvantage of porous materials of conventional composition is however that the already minimal mechanical load capacity and biomechanical compatibility with bone is further reduced. Therefore, for the treatment of larger bone defects especially the combination of open-cell metal structures with porous (organo) mineral fillers is advantageous and expressly to be understood as a part of the present invention. In view of the great variety of clinically existing bone defects, there is also a broad demand profile with respect to the design of the bone implants according to the invention. In this connection, the porosity of the bone replacement material is a parameter by means of which in particular bioactivity and resorption rate (and in combination with active ingredients also their release rate) can be affected.

Porous bone replacement materials can be obtained in that gas formers or dissolvable particles are added to the preparation or in that in case of cement-like preparations the cement reaction itself entails gas formation (Del Real R P, Wolke J C M, Vallet Regi M, Jansen J A (2002): A new method to produce macropores in calcium phosphate cements; Biomaterials 23:3673-3680). Especially advantageous, and in the context of the above description preferred with respect to the present invention, are cement-like fillers that fill the pore system of the open-cell metal structure to less than 80%, preferably to less than 70% and especially preferred to less than 50% of the theoretically possible degree of filling. Also preferred is an interconnectivity of the remaining pore system within the cement-like filler. This is achievable, for example, by infiltration of the open-cell metal structure with a cement slurry and subsequently blowing out excess material while the remaining filler subsequently is cured under controlled conditions. By a targeted adjustment of the cement slurry consistency and the conditions for removal of the excess material the remaining pore volume can be adjusted within a wide range and in particular in the preferred range (see above). In the context of the invention an interconnectivity of approximately 50% of the remaining pore system is entirely satisfactory for a fast bone integration. It therefore has an interconnectivity of the remaining pore system of >25%, preferably >40%, and especially preferred >50%.

In contrast to dense ceramic implant materials and in view of the goal of a high degree of material exchange with the body liquid, a porosity of the bone replacement material of preferably >20% and in particular preferred >50% is preferred so that the preparation of at least one bone replacement material fills the accessible pore volume of the pore system of the open-cell metal structure, relative to the theoretical/calculated possible degree of filling and calculated based on the dry matter, to less than 80 weight percent and in particular preferred to less than 50%.

On the other hand, a preferred embodiment of the invention is comprised of a composition in which the preparation of at least one bone replacement material fills at least 1% and especially preferred at least 5% of the accessible pore volume of the open-cell metal structure relative to the theoretical/calculated possible degree of filling and calculated based on dry matter.

According to claim 16, the open-cell metal structure has a compression strength of >1 MPa and <50 MPa, the preparation of at least one resorbable bone replacement material alone has a compression strength of >2 MPa, and the combination of both components has a compression strength that is greater than the sum of the compression strengths of both components.

In a special embodiment of the invention the filler itself has a relatively high strength (compression strength >20 MPa) and is at the same time resorbable by bone cells and is capable of remodeling. In this case, the open-cell metal structure, in analogy to steel-reinforced concrete, takes on the role of reinforcement so that the compression strength of the cement-bike bone replacement material (with minimal own flexural strength) may be combined in an advantageous way with the flexural strength of the metal structure (that itself as a result of its high porosity has a relatively minimal compression strength). In particular, when using bio-corrodible metals as a reinforcement, for the first time a bone replacement material can be provided in this way that combines a high mechanical (instant) load capacity with high biological activity and complete remodeling capability or resorption capability. The excellent compatibility of the cement-like (calcium phosphate) bone replacement materials with all relevant active ingredients enables moreover the simultaneous utilization of the implant as a local drug delivery system for active ingredients for stimulating bone healing and bone buildup and for avoiding side effects (see above).

According to the invention in this embodiment open-cell metal structures, optionally with graduated porosity depending on the envisioned field of application, with a compression strength of >1 MPa and <10 MPa are combined with a bone replacement material that itself has a compression strength of >2 MPa and in which the compression strength of the combination of both components is >12 MPa. This embodiment is indicated in particular for filling spongiosal bone defects with relative minimal density and strength in which primarily a fast regeneration is important.

In a further preferred embodiment an open-cell metal structure, optionally with graduated porosity depending on the envisioned field of application, with a compression strength of >1 MPa and <20 MPa is combined with a bone replacement material that itself has a compression strength of >5 MPa and in which the compression strength of the combination of both components is >25 MPa. This embodiment is suitable in principle for the repair and filling of all spongiosal bone defects and in particular those with relatively high density and strength. In this context, this embodiment can be viewed as a universal filler for bone defects under load in spongiosal bones.

In a further preferred embodiment an open-cell metal structure, optionally with graduated porosity depending on the envisioned field of application, with a compression strength of >5 MPa and <45 MPa is combined with a bone replacement material that itself has a compression strength or strength of >5 MPa and in which the compression strength of the combination of both components is >50 MPa. This embodiment is suitable also for repair of cortical bone defects or such bone defects that have spongiosal as well as cortical parts.

According to a further advantageous embodiment of the invention according to claim 17, the open-cell metal structure is comprised of a macroscopically homogenous or a graduated pore system in which only a portion of the pore system is filled with a preparation of at least one resorbable bone replacement material and the remaining portion of the pore system remains either unfilled or the remaining portion of the pore system is partially or entirely filled with a metal-based, ceramic-based or a polymer-based material.

As already mentioned above, open-cell metal structures with bioactive filler can be of a macroscopically homogeneous construction, can have a graduated structure, or may be a component of an implant of a more complex build. According to the invention in all cases at least one portion of the open-cell metal structure is loaded with a preparation of at least one bone replacement material in one of the aforementioned ways. Even as a component of a complexly built implant such as a joint prosthesis, the open-cell metal structure with a bioactive filler fulfills the function of the biologic and biomechanical stimulation of bone growth and improves thus the conditions for a permanent implant integration.

According to claim 18 the bone implant contains for this purpose at least one partially filled metal structure and additional structures that are fixedly connected thereto which additional structures are substantially dense or have a porosity that is by a factor >10 smaller than that of the open-cell metal structure and, in turn, that is comprised of a metal-based, ceramic-based or a polymer-based material.

In the simplest case, such a complexly structured implant is comprised of an open-cell metal structure that has a macroscopically homogeneous or a graduated pore system, in which only a portion of the pore system is filled with a preparation of at least one bone replacement material and the remaining portion of the pore system remains either unfilled or this portion of the pore system is partially or entirely filled with a metal-based, ceramic-based, or polymer-based material that is comprised of non-resorbable and/or non-bioactive material.

In a modification, the bone implant according to the invention of an open-cell metal structure and a bioactive filler is combined with further implant structures that are substantially dense or have a porosity that is smaller by at least a factor >10 than that of the open-cell metal structure and that themselves are comprised of a metal-based, ceramic-based, or a polymer-based material, in such a way that all implant components at the time of implantation are connected fixedly with one another. This combination and fixed connection can be realized already in the context of industrial manufacture or immediately before implantation. In the latter case, this provides the possibility of a modular implant construction in which the user in accordance with the individual situation, in particular in accordance with the size and shape of the bone defect to be filled, for example, in the context of revision of a joint prosthesis, may select a matching element comprised of an open-cell metal structure and a bioactive filler and by means of suitable connecting devices may connect it to the remaining implant component(s). The connection can be realized, for example, by means of screw connections wherein in this case the open-cell metal structure advantageously contains reinforcements that prevent damage of the open-cell metal structure during the screwing action.

In certain clinical situations it can moreover be very expedient that the implant according to the invention is brought only immediately before or during operation into its final shape or composition. This aspect is especially important in such cases where the surgeon wants to or must adjust the open-cell metal structure or the assembled implant as a whole in accordance with the shape and size of the bone defect and optionally must mechanically work for this purpose the implant and subsequently clean it. Processing as well as particularly subsequent cleaning steps can damage the bioactive filler and, in case of incorporated active ingredients, can even make them completely unusable. Moreover, it can be expedient and necessary to combine prior to or during the operation the open-cell metal structure with a specially selected preparation of at least one bone replacement material. This may be necessary for mechanical reasons because the surgeon may be able realize only during surgery what type of the mechanical requirements will be put on the implant. A further reason may be the necessity for a combination with a special pharmacologically active ingredient in order to enhance thus the function of the implant. Industrially, only a few bone replacement materials and active ingredients are offered in combination. On the other hand, the bioactive fillers according to the invention are compatible with and combinable in a simple way with a variety of active ingredients.

An important aspect of the invention is therefore providing a set of components, containing at least one open-cell metal structure and a pasty or cement-like composed bioactive bone replacement material or a composition from which a pasty or cement-like preparation of at least one bone replacement material can be produced, with which before or during surgery a bone implant can be produced in accordance with one of the preceding claims. Into these pasty or cement-like bioactive preparations suitable active ingredients may then be introduced in accordance with clinical requirements.

According to claim 19 the set is comprised of components of at least one open-cell metal structure or a bone implant that contains an open-cell metal structure and a pasty or cement-like composed preparation of at least one bone replacement material or a composition from which a pasty or cement-like preparation of at least one bone replacement material can be produced. According to an advantageous embodiment according to claim 20 this set is made available in sterilized form.

For a simple and reproducible loading of the open-cell metal structure with the preparation of at least one bone replacement material further auxiliary means may be required. This encompasses devices for mixing cement-like compositions of powder and liquid (and optionally admixture of active ingredients) as well as suitable instruments for processing and securing the metal structures, optionally vessels and devices for cleaning after processing, and syringes and cannulas (or other application devices) for the injection of the pastes or cements into the open-cell metal structure. Providing special sets for a targeted preparation of bioactive fillers for the subsequent loading of open-cell metal structures is therefore also an important aspect of the invention, in particular when these sets contain components that are matched to one another, are packaged together and/or are made available in sterilized form.

According to an advantageous embodiment of the invention according to claim 21 the open-cell metal structure has fill openings and/or further devices for filling with the preparation of at least one resorbable bone replacement material of a non-metallic component in order to prepare before or during operation bone implants according to the invention in a simpler way.

In addition to specialized devices for loading the open-cell metal structures, also special structures in the open-cell metal structures themselves can facilitate or even make possible at all the pre-surgery or intra-surgery loading with the preparations of at least one bone cement replacement material. This holds true in particular for small-pore metal foams, those with (hollow) sphere structure and those with relatively minimal total porosity, but also generally for large volume implants. The structures that are useful for loading with bioactive fillers are comprised primarily of bores or other recesses that enable injection of the preparation by means of a syringe or loading from the exterior. In this connection, the appropriate assisting structures and the methods of loading can be embodied in various ways (like the bone implants according to the invention themselves and the compositions of the bone replacement materials). Decisive in the context of the invention is however that the appropriately structured open-cell metal structure contains these structures primarily for the purpose of loading with the preparation of at least one bone replacement material.

An aspect of the invention is also the use of open-cell metal structure with interconnected pore system for producing a bone implant according to the invention as well as the use of the preparation of at least one resorbable bone replacement material in combination with the open-cell metal structure for producing a bone implant according to the invention. The open-cell metal structure as well as the preparation of at least one resorbable bone replacement material are constructed for this purpose as explained above.

### EMBODIMENTS

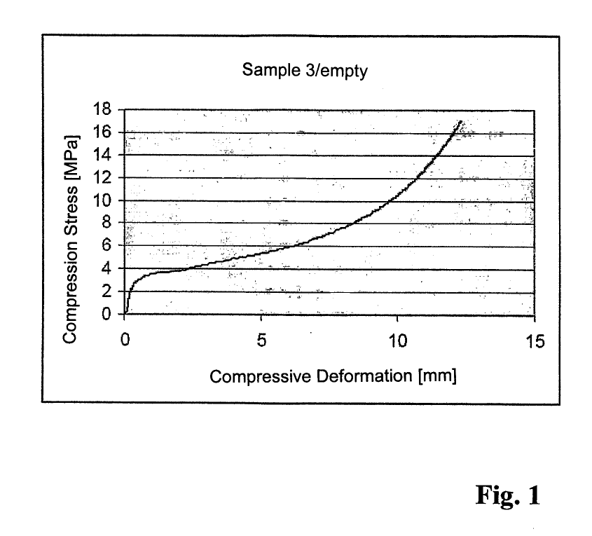

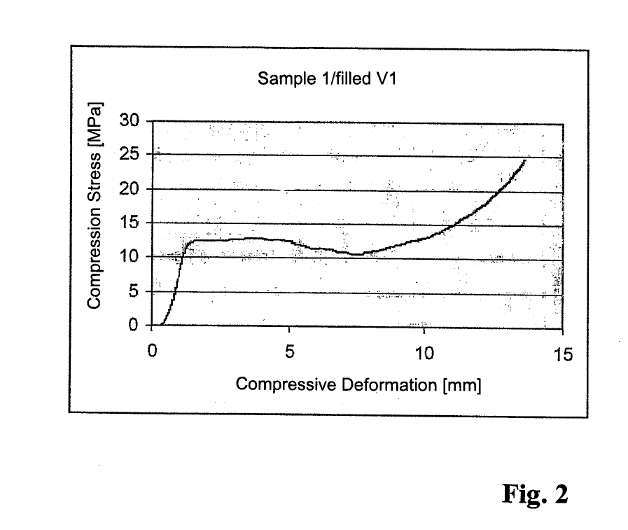

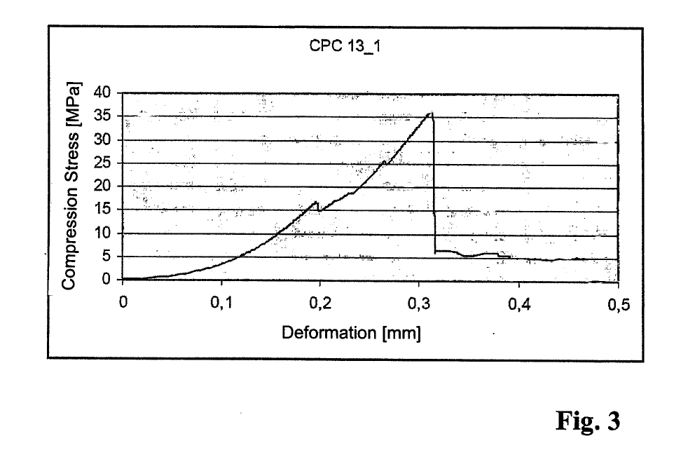

Based on the attached diagrams embodiments of the invention will be explained. In this connection, FIG. 1 shows the deformation diagram of an unfilled metal foam on the basis of iron, FIG. 2 the deformation diagram of an iron-based metal foam filled with a calcium phosphate cement preparation, wherein the employed calcium phosphate cement preparation has its own compression strength of 2 MPa. FIG. 3 shows the deformation diagram of a commercial calcium phosphate cement with a compression strength of approximately 36 MPa. FIGS. 1 through 3 show that the commercial calcium phosphate cement upon very minimal deformation fails catastrophically in that the sample breaks up into many individual pieces. In contrast to this, the composite material according to the invention, despite a much smaller compression strength of the individual components, has a significantly increased compression strength in comparison to iron-based metal form and a high deformability without dramatic drop in strength.

### Example 1

### Bone Implant on the Basis of a Metal Foam Filled with Nano-Crystalline Hydroxyl Apatite

A metal foam of implant steel (360 L) is used that is produced by Fraunhofer Institut für Verfahrenstechnik and angewandte Materialforschung (IFAM Dresden, Adler et at, Sintered Open-Celled Metal Foams Made by Replication Method—“Manufacturing and Properties on Example of 316L Stainless Steel Foams”, in Cellular Metals and Polymers 2004, edited by R. F. Singer, et al, and John Banhart “Manufacture, characterisation and application of cellular metals and metal foams”; in Progress in Materials Science 46 (2001) 559-632) according to a powder-metallurgical method (pore size approximately 45 ppi/“pores per inch”). As a nano-crystalline hydroxyl apatite preparation a pasty preparation with 35% solids contents (in aqueous preparation) is employed.

The metal foam (diameter 3 cm, height 1 cm) is positioned on a filter paper above a glass frit in a tightly fitting plastic pipe (height 3 cm). Onto the metal foam the preparation of nano-crystalline hydroxyl apatite (nano-HA) is applied in a quantity that surpasses minimally the pore volume of the metal foam and is distributed uniformly about the metal foam cylinder. Subsequently, a tightly sealing piston (with venting device) is inserted into the plastic pipe and the nano-HA preparation is pressed into the metal foam until it has reached the filter paper. The filled metal foam cylinder is removed and the filter paper and excess nano-HA preparation are removed. This cylinder is subsequently packaged by vacuum-sealing in a plastic film and the thus prepared bone implant is ready for use after sterilization.

While the unreinforced hydroxyl apatite preparation has no strength on its own and the open-cell metal structure is not bioactive, the combination product provides a highly bioactive material that in particular exhibits an excellent biomechanical adaptation to osteoporotic bone and immediately after implantation can be subjected to loads. Fields of application are defect fractures and bone buildup in osteoporotic bone in which non-bioactive implants cannot be incorporated satisfactorily with respect to bone growth and stiff implants would mechanically damage the adjoining bone. Preferred uses are vertebra fusions in case of pronounced osteoporosis.

### Example 2

### Bone Implant of the Basis of Metal Foam Filled with Calcium Phosphate Cement

The same metal foam as disclosed in Example 1 is used. As a filler calcium phosphate cement of own production with the following composition is used:

### Powder Component:

60% TCP (fired at 1300 ground to a particle size of <20 μm)
26% calcium hydrogen phosphate (anhydrous CaHPO₄
10% calcium carbonate (CaCO₃)
4% hydroxyl apatite (precipitated),
   all components are intensively mixed and ground together.

As a mixing solution, to a 2% sodium hydrogen phosphate (Na₂HPO₄) solution phosphoserine in a concentration of 50 mmol/l is added and subsequently the pH value is adjusted to 8.5. Subsequently, the powder component is homogeneously mixed with this mixing solution in a powder/liquid ratio of 0.7 at a temperature of 10° C. The obtained paste is uniformly applied onto the metal foam in an experimental set-up according to the Example 1 (without filter paper and glass frit in an airtightly sealable plastic pipe) and subsequently is introduced into the metal foam on a shaker plate of a sieving machine with evacuation of the plastic cylinder. After one minute the entire pore volume of the metal foam cylinder is filled with the calcium phosphate cement without macroscopically visible pores. Excess calcium phosphate cement is removed after removal of the loaded metal foam from the plastic pipe and the metal foam is then cured for final curing of the calcium phosphate cement in an incubator at 37° C. with water vapor saturation for 72 hours. Subsequently, the loaded metal foam is then dried until a constant weight is reached at 40° C. and 0.1 bar, is packaged, sterilized and is thus ready for use.

The thus obtained implant material has a higher mechanical load capacity while having also a high bioactivity in comparison to the material of Example 1. Preferred fields of application are also vertebra fusions and additionally a vertebra replacement with biomechanical adaptation to the surrounding bone. In this case, the cement-like filler contributes to initial strength wherein with increasing resorption and bony replacement, the mechanical loading is increasingly taken over by the bone.

### Example 3

### Bone Implant on the Basis of a Hollow Sphere Metal Structure Filled with Nano-Crystalline Hydroxyl Apatite

As a porous metal structure cylinders of a diameter of 3 cm, height of 1 cm, are used that are comprised of hollow metal spheres, sintered together, with a size of the individual spheres of 1 mm in diameter. The employed material is implant steel (316 L) produced by Fraunhofer Institut für Verfahrenstechnik and angewandt Materialforschung (IFAM Dresden) in accordance with a powder-metallurgical method. In the same experimental set-up as in 1, a paste of nano-crystalline hydroxyl apatite with a solids content of 35% is introduced into the pore system of the metal cylinder. After loading the same procedure as in Example 1 is followed.

### Example 4

### Bone Implant on the Basis of a Hollow Sphere Metal Structure Filled with Calcium Phosphate Cement

As a porous metal structure the same metal structure is used as in Example 3. Subsequently, the same calcium phosphate cement as used in Example 2 is introduced into the pores of the hollow sphere metal structure and the procedure as disclosed in Example 2 is followed.

The implant materials according to Examples 3 and 4 have a higher mechanical load capacity than the preceding examples. The bioactivity is comparable. The preferred fields of application are bone defects in patients with less severe osteoporosis.

### Example 5

### Bone Implant on the Basis of a Porous Metal Structure of Pure Titanium Filled with Nano-Crystalline Hydroxyl Apatite

A porous metal structure of pure titanium with a diameter of 7 mm, a height of 10 mm, and a regular pore arrangement (pore size approximately 350 μm) is produced according to the method of direct laser shaping by Fraunhofer Institut für Lasertechnik (ILT in Aachen) from pure titanium powder and made available for the loading test. Loading with nano-crystalline hydroxyl apatite is carried out in analogy to Example 1. The result shows complete macroscopically homogenous loading.

### Example 6

### Bone Implant on the Basis of Porous Metal Structure of Pure Titanium Filled with Calcium Phosphate Cement

As a porous metal structure a cylinder of pure titanium according to Example 6 is employed. Loading with calcium phosphate cement is done in analogy to Example 2. The result corresponds to that of Example 2.

Examples 5 and 6 show the transferability onto further typical metallic implant materials that are widely used in bone surgery.

### Example 7

### Bone Implant on the Basis of an Iron-Based Metal Foam Filled with Nano-Crystalline Hydroxyl Apatite

A metal foam on the basis of iron is used that is provided by Fraunhofer Institut für

Verfahrenstechnik and angewandte Materialforschung (IFAM Dresden), produced in accordance with a powder-metallurgical method (pore size approximately 45 ppi).

Loading with nano-crystalline hydroxyl apatite is realized in analogy to Example 1 with the same loading result.

### Example 8

### Bone Implant on the Basis of an Iron-Based Metal Foam Filled, with Calcium Phosphate Cement

An iron-based metal foam is used that is supplied by Fraunhofer Institut für Verfahrenstechnik and angewandte Materialforschung (IFAM Dresden) and produced by a powder-metallurgical process (pore size approximately 45 ppi).

As a filler a calcium phosphate cement of the following composition is used: 60% by weight α-TCP, 26% by weight calcium hydrogen phosphate, 10% by weight calcium carbonate, 4% by weight hydroxyl apatite are comminuted to fine powders and homogeneously mixed. 10 g of this powder mixture are homogeneously mixed with 7 ml of a 2% sodium hydrogen phosphate solution to a low-viscosity paste at a temperature of 10° C. The obtained paste is applied in an experimental set-up in an analogy to 1 (without filter paper and glass frit in an airtightly closable plastic pipe) uniformly onto the metal foam and subsequently introduced into the metal foam by using a shaker plate of a sieving machine with evacuation of the plastic cylinder. After one minute the entire pore volume of the metal foam cylinder is filled with the calcium phosphate cement without macroscopically visible pores. Excess calcium phosphate cement is removed after removal of the loaded metal foam from the plastic pipe and the metal foam is then cured for final curing of the calcium phosphate cement in an incubator at 37° C. and with water vapor saturation for 72 hours. Subsequently, the loaded metal foam is dried until a constant weight is reached at 40° C. and at 0.1 bar. Three cylinders each of the diameter 10 mm and height 20 mm are then tested in the filled as well as unfilled state on a material testing machine of the company Instron type 5566 (10 kN) with a feed rate of 1 mm/min with respect to compression strength. The unfilled samples achieve on average a compression strength value of approximately 3.0 MPa up to the limit of elastic deformation (see FIG. 1) while the samples filled with calcium phosphate cement reach a comparative value of approximately 12 to 20 MPa (see FIG. 2).

FIG. 1 shows that the elastic deformation of the unfilled metal foam at a compression stress of approximately 3 MPa passes into a plastic deformation. FIG. 2 shows that the elastic deformation of the metal foam that is filled with calcium phosphate cement preparation passes into a plastic deformation only once a compression stress of approximately 12 MPa has been reached. A compression stress remains almost constant across a large deformation range and then increase farther. In contrast to this the compression stress for a shaped body of unreinforced commercially available calcium phosphate cement drops catastrophically after reaching the failure limit (see FIG. 3).

FIG. 3 shows the pressure deformation of a typical calcium phosphate cement (without reinforcement) with the comparatively high maximum compression strength of approximately 36 MPa. Already for a minimal deformation of approximately 0.3 mm (corresponding to <2%) a catastrophic strength loss with complete destruction of the sample occurs. A plastic deformation range practically does not exist. Accordingly, unreinforced calcium phosphate cements are unsuitable for load-bearing applications. The same holds true for sintered bone ceramics wherein however it is even more important that such materials are incapable of following a physiological force introduction in any way.

The samples filled in accordance with Example 8 show a de formation behavior that, despite the relatively minimal compression strength of the unfilled metal foam, differs significantly from the typical deformation behavior of an unreinforced calcium phosphate cement (compare compressive deformation of a typical calcium phosphate cement; FIG. 3). These test results show thus in an exemplary fashion that a reinforcement of bone replacement materials with open-cell metal foams leads to combination products with novel biomechanical properties.

Metal foams on the basis of iron are considered corrodible under implantation conditions wherein the corrosion products should be biologically compatible. The preferred fields of use are primarily in younger patients where a complete resorption of the implant material is desired. In accordance with the adjustable resorption times (very high for suspensions of nano-crystalline hydroxyl apatite, slower and adjustable in case of cement-like preparations), first the filler material is resorbed and replaced with bone while subsequently the iron foam is corroded and within a time period of 6 months to approximately 3 years is degraded. As a result of the increasing mechanical loading, the grown-in bone is stimulated to build up additional bone substance and to reinforce itself. The combination material fulfills thus (for the first time) the goal of immediate load-bearing capacity of the implant, a high bioactivity (that is not provided by the pure metal structures), and a complete resorption capability with simultaneously increasing biomechanical bone stimulation.

### Example 9

### Bone Implants on the Basis of a Metal Foam Filled with Mineralized Collagen

Metal foam according to Example 1 is loaded in the same experimental set-up as in Example 1 with demineralized collagen. Collagen is produced in accordance with the method described by Gelinsky et al. in Mat-wiss, Werkstofftech. 35, No. 4, 229-233. After the loading step of the metal foam with the suspension of the mineralized collagen the sample is frozen at −20° C. and subsequently lyophilized. The obtained sample is thereafter macroscopically homogenous and filled with a lyophilisate of mineralized collagen.

The principally highly bioactive materials of mineralized collagen without their own structural strength can be further developed by combination with the open-cell metal foams to mechanically loadable implant materials and are thus suitable in a special way as cell carriers for tissue engineering.

### Example 10

### Bone Implants on the Basis of a Metal Foam Filled with Calcium Phosphate Cement and Residual Porosity

In analogy to the Examples 2, 6, and 8 a metal foam on the basis of iron with a porosity of approximately 90% and a pore size of approximately 30 ppi (produced by Fraunhofer Institut für Verfahrenstechnik and angewandte Materialforschung (IFAM Dresden)) is infiltrated with calcium phosphate cement. The sample (diameter 30 mm, height 10 mm) is loaded in the air-tightly sealed device at one side slowly with (air) overpressure (water vapor saturated air) and in this way a portion of the cement paste is pressed out of the pore system. The quantity of remaining cement is determined by gravimetry and is approximately 50% of the value that is reached in the method in analogy to Example 8 for complete loading with the same calcium phosphate cement and a metal foam of this porosity.

### Example 11

### Bone Implant on the Basis of a Metal Foam Filled with Calcium Phosphate Cement and Residual Porosity

In analogy to the Examples 2, 6, and 8 a metal foam on the basis of iron with a porosity of approximately 90% and a pore size of approximately 30 ppi (produced by Fraunhofer Institut für Verfahrenstechnik and angewandte Materialforschung IFAM Dresden) is infiltrated with calcium phosphate cement. Through the sample (diameter 30 mm, height 10 mm), in the tightly closed device slowly a neutral oil (Migliol) is forced through. After curing of the cement (72 hours at 37° C.) the sample is dried until a constant weight is reached and is washed several times with acetone in order to remove the adhering neutral oil. By gravimetry, the residual filler is calculated to 60% of the initial value and 41% of the theoretically possible degree of filling. The interconnectivity of the pore system is determined microscopically to a value of approximately 70%.

The Examples 10 and 11 result in materials that contain highly bioactive fillers and at the same time exhibit an interconnected pore system. In this way, the bone can grow particularly fast into the implant material. This combination enables for the first time the targeted utilization of the high bioactivity of nano-crystalline calcium phosphates in combination with interconnected pore systems (that enable an intensive exchange of materials with the surrounding medium) and at the same time provides biomechanics adapted to the field of application with complete resorption capability.

What is claimed is:

1.-23. (canceled)

24. A bone implant comprising:
an open-cell metal structure with an interconnected pore system;
wherein a stiffness of the open-cell metal structure is significantly lower than that of a solid material consisting of the same metal as the open-cell metal structure;
wherein the stiffness of the open-cell metal structure surpass the stiffness of healthy cortical human bone by not more than a factor 2;
a preparation comprising at least one bone replacement material, wherein the pore system is filled at least partially with the preparation, wherein at least one bone replacement material is present in cells of the open-cell metal structure as a powder, a pasty suspension, a lyophilisate, or a cement-like cured material.

25. The bone implant according to claim 24, wherein the open-cell metal structure is comprised of stainless steel, cobalt-based alloys, pure titanium, titanium alloys, tantalum, tantalum alloys, niobium, Nitinol, gold or silver.

26. The bone implant according to claim 24, wherein the open-cell metal structure is comprised of a metal or an alloy of said metal that is corrodible (bio-corrodible) under biological conditions and wherein corrosion of said metal or a main component of sad alloy taking place under biological conditions produces compounds that occur naturally in a body of a vertebrate.

27. The bone implant according to claim 24, wherein the pore system of the open-cell metal structure is a graduated pore system in which at least one of a pore diameter, a pore shape, and a pore volume is changed in at least one section plane of the open-cell metal structure.

28. The bone implant according to claim 24, wherein the at least one bone replacement material comprises nano-crystalline calcium phosphate or forms nano-crystalline calcium phosphate after implantation.

29. The bone implant according to claim 24, wherein the preparation comprises composite materials that contain collagen, gelatin and/or other proteins of the organic extracellular matrix of the bone and further contain as mineral components phosphates, silicates, carbonates or sulfates of alkali and earth alkali elements or ammonium compounds or combinations thereof.

30. The bone implant according to claim 24, wherein the preparation contains biologically and/or pharmacologically active ingredients.

31. The bone implant according to claim 24, wherein the preparation is porous and wherein the preparation fills an accessible pore volume of the pore system of the open-cell metal structure, relative to the theoretical/calculated possible degree of filling and calculated based on dry matter, to 5 to 80 weight percent

32. The bone implant according to claim 24, wherein the open-cell metal structure has a compression strength of >1 MPa and <50 MPa, wherein the preparation alone has a compression strength of >2 MPa, and wherein a combination of the open-cell metal structure and the preparation has a compression strength that is greater than the sum of the compression strengths of components of the open-cell metal structure and the preparation.

33. The bone implant according to claim 24, wherein the pore system of the open-cell metal structure is macroscopically homogenous or graduated, wherein only a portion of the pore system is filled with the preparation and wherein the remaining portion of the pore system is either unfilled or the remaining portion of the pore system is filled with a metal-based, ceramic-based, or polymer-based material either completely or partially.

34. The bone implant according to claim 24, comprising additional structures that are fixedly attached, wherein the additional structures are substantially dense or have a porosity that is by a factor of >10 smaller than that of the open-cell metal structure and are comprised of a metal-based, ceramic-based, or polymer-based material.

35. A set comprising;
at least one open-cell metal structure;
a pasty or cement-like preparation of at least one bone replacement material or a composition from which the pasty or cement-like preparation of at least one bone replacement material is producible,
wherein, before or during operation, a bone implant according to claims 24 is produced with the set.

36. A bone implant comprising at least one open-cell metal structure that has fill openings and/or devices for filling with a preparation of at least one bone replacement material.

37. A method for producing a bone implant, comprising the steps of:
providing an open-cell metal structure with an interconnected pore system, wherein a stiffness of the open-cell metal structure is significantly lower than that of a solid material consisting of the same metal as the open-cell metal structure;
filling the pore system of the metal structure with at least one preparation comprising at least one bone replacement material.
